(19) United States
(12) Patent Application Publication
(10) Pub. No.: US 2010/0035977 A1
(43) Pub. Date: Feb. 11, 2010

(54) TWO-COMPONENT TAXANE CONTAINING PHARMACEUTICAL COMPOSITION

(76) Inventor: Vladimir Kysilka, Brno (CZ)

Correspondence Address: LADAS & PARRY LLP, 26 WEST 61ST STREET, NEW YORK, NY, 10023, US

(21) Appl. No.: 12/522,329

(22) PCT Filed: Oct. 19, 2007
(86) PCT No.: PCT/CZ2007/000092

§ 371 Date: Oct. 19, 2009

(30) Foreign Application Priority Data
Jan. 23, 2007 (CZ) PV 2007-56

(51) Int. Cl. A61K 31/337

(52) U.S. Cl. 514/449

(57) ABSTRACT

A stabilized, injectable pharmaceutical composition for human administration comprises (a) an antineoplastic compound, (b) a solubilizing/dispersing agent, and (c) a stabilizing amount of an anti-oxidant.

### FIELD OF THE INVENTION

The present invention relates to a two-component taxane containing pharmaceutical compositions useful for the preparation of infusion solutions which can be used for the treatment of cancer. Said pharmaceutical compositions are stable, easy to prepare and easy to use. The invention further relates to a method for the preparation of infusion solutions comprising taxane derivatives.

### BACKGROUND OF THE INVENTION

Pharmaceutical compositions comprising taxane derivatives, e.g. paclitaxel, docetaxel, ortataxel or protaxel, are widely used for the therapy of cancers. The most frequently used taxane derivatives are paclitaxel and docetaxel. Taxane derivatives have generally very poor water solubility, which complicates their formulation into pharmaceutical compositions useful for the preparation of taxane containing infusion solutions.

It is known that some surface active agents (surfactants) are capable of keeping taxane derivatives in solution when diluted by a common infusion solutions, such as isotonic sodium chloride or glucose based solutions, but unfortunately, the dissolution of taxane derivatives in surfactants, themselves, is difficult. This difficulty was alleviated by the use of a co-solvent system comprising a suitable surfactant and ethanol. Thus, for the preparation of a taxane containing infusion solution a pharmaceutical composition comprising 6 mg of paclitaxel per 1 ml of a co-solvent system comprising 1:1 v/v mixture of polyoxyethylated castor oil and ethanol was proposed (Rowinsky et al.: Journal of National Cancer Institute, vol. 82, No. 15, p. 1247-1259, 1 Aug. 1990). A widely used commercial product comprising paclitaxel as an active ingredient, Taxol®, is derived just from the above mentioned composition. Said composition has however two serious drawbacks. The first one is a severe side effect of polyoxyethylated castor oil. This drawback can be alleviated by premedicating patients with steroids and antihistaminics but, nevertheless, the risk of anaphylactic shock still remains. The second drawback is the general instability of taxane derivatives in co-solvent systems comprising a polyoxyethylated surfactant and ethanol. Said instability is caused by the traces of impurities comprised in polyoxyethylated surfactants, e.g. polyoxyethylated castor oil (polyoxyethyleneglycerol triricinoleate, Cremophor EL). In the presence of ethanol these impurities decompose taxane derivatives very quickly. A problem of said instability of taxane derivatives in co-solvent systems comprising a polyoxyethylated surfactant and ethanol has been approached in many patents or patent applications.

The above mentioned drawbacks were partially solved by a composition comprising a taxane derivative in a co-solvent system comprising polysorbate and ethanol. Polysorbate, as a surfactant, has less side effects than polyoxyethylated castor oil, a problem of poor solubility of taxane derivatives in polysorbate, itself, however remains. In order to overcome the last mentioned problem the use of a sufficient amount of ethanol for taxane derivative dissolution in polysorbate was proposed but then the added ethanol had to be removed by distillation in order to minimize the risk of taxane derivatives decomposition in the resulting co-solvent system. A pharmaceutical composition of taxane derivatives in a co-solvent system comprising polysorbate and ethanol wherein the content of ethanol is less than 5% is claimed in EP 0 593 601. This composition is relatively stable but it is poorly miscible with common infusion solutions due to a low content of ethanol, which causes gel formation and foaming during the preparation of a taxane loaded infusion solution. This problem was alleviated by premixing a low ethanol taxane and polysorbate containing composition with an aqueous solution of a dilution additive, e.g. ethanol, prior to the dilution of the former composition to form the resulting infusion solution. In this way, gel formation can be prevented and foaming can be reduced. A pharmaceutical composition, having a double compartment, intended for the preparation of a solution for infusion which is composed by the solution of a taxane derivative in polysorbate containing less than 5% of ethanol and by the solution of 13% (w/w) of ethanol in water wherein these two solutions are mixed together before the preparation of a solution for infusion is claimed in EP 0 671 912. This double compartment pharmaceutical composition is the basis of a widely used docetaxel commercial product which is sold under the trade name Taxotere®.

The above mentioned compositions comprising taxane derivatives in polysorbate with a low content of ethanol have several drawbacks. The sterilisation by ultrafiltration of a highly viscous polysorbate solution containing a taxane derivative is very difficult. The high viscosity also makes difficult metering exact dosages of the polysorbate solution and brings about high losses of a very expensive taxane derivative during the filling procedure. There is also a risk of possible partial decomposition of a taxane derivative when ethanol is distilled off from the co-solvent system comprising a taxane derivative, polysorbate and ethanol.

The drawbacks of the above mentioned prior art are removed by the present invention.

### DESCRIPTION OF THE INVENTION

The present invention provides a two-component taxane containing pharmaceutical composition useful for-the preparation of infusion solutions wherein decomposition and/or precipitation of taxane derivatives from the infusion solutions is avoided, said composition comprising a) taxane derivative solution in ethanol and b) a solution of a surfactant selected from polysorbates or polyoxyethylated castor oil (polyoxyethyleneglycerol triricinoleate) in ethanol or a mixture of ethanol and water.

The invention further provides a method for the preparation of an infusion solution comprising taxane derivatives, which comprises
i) mixing a) a taxane derivative solution in ethanol and b) a solution of a surfactant selected from polysorbates or polyoxyethylated castor oil (polyoxyethyleneglycerol triricinoleate) in ethanol or a mixture of ethanol and water and then
ii) diluting the premix from step i) with a common unloaded infusion solution.

We have found that solution a) of a taxane derivative in ethanol is easy to prepare, easy to handle, and it can be easily metered and filled into suitable containers with substantially lower losses of an expensive taxane active product. Moreover, ethanolic solutions of taxane derivatives are very stable during a prolonged time period. Said solutions can be easily prepared by dissolving taxane derivatives in ethanol at room temperature or at a mildly elevated temperature with subsequent ultrafiltration and filing the solution into suitable containers. A preferred solution a) of a taxane derivative in ethanol is a docetaxel solution in ethanol having the concentration in the range of from about 10 to about 100 mg/ml, preferably from about 40 to about 80 mg/ml. Another preferred solution a) of a taxane derivative in ethanol is a paclitaxel solution in ethanol having the concentration in the range of from about 10 to about 25 mg/ml, preferably from about 15 to about 20 mg/ml.

A preferred surfactant of component b) is a polysorbate, in particular Polysorbate 80. Polysorbate 80 is polyoxyethylene 20 sorbitan monooleate, CAS number [9005-65-6]. Although polyoxyethylated castor oil (polyoxyethyleneglycerol triricinoleate), such as Cremophor EL, can be also used as a surfactant, polysorbates and particularly Polysorbate 80 are highly preferred since they provide the best results from the point of view of physical stability of taxane derivatives in infusion solutions. Solutions of Polysorbate 80 in ethanol or in a mixture of ethanol and water are less viscous, sufficiently stable and more suitable for filling and handling procedures. Although, in general, any ethanolic or aqueous-ethanolic solvent of component b) could be omitted, and thus, a surfactant alone could be used as component b), this alternative is not preferred due to the fact that in this instance step i) would have to be carried out at elevated temperatures in order to compensate for the higher viscosity caused by the absence of any solvent in component b). The possibility of operating at mild temperatures, which is one of the advantages of the present invention, would thus have to be sacrificed.

Surfactant solutions b) can be easily prepared by mixing a selected surfactant with ethanol and optionally adding water. The resulting solution is then ultrafiltered and filled into suitable containers. The recommended amount of Polysorbate 80 is the amount necessary for preventing precipitation of a taxane derivative from an infusion solution. The preferred concentration of a taxane derivative is about 1 mg/ml. We have found that an amount of 20-25 μl Polysorbate 80 per 1 mg of docetaxel is sufficient. When paclitaxel is used instead of docetaxel, the amount of Polysorbate 80 must be at least doubled, i.e. it should be at least 40-50 μl per 1 mg of paclitaxel.

A two-component taxane containing pharmaceutical composition according to the present invention can be used for the preparation of infusion solutions. As mentioned above, a method for the preparation of said infusion solution comprises i) mixing component a) as identified above with component b) as identified above and then ii) diluting the premix from step i) with a recommended amount of a common unloaded infusion solution, to obtain a solution for infusion containing taxane derivatives having a recommended therapeutic concentration of an active ingredient for cancer therapy. The above mentioned solutions a) and b) are preferably mixed shortly or just before the preparation of an infusion solution to avoid problems with the instability of taxane derivative during prolonged time periods. Once the infusion solution has been prepared, it should be preferably used without any delays.

A two-component taxane containing pharmaceutical composition according to the present invention is simple, stable, easy to prepare, easy to handle and it is suitable for the preparation of taxane derivative containing infusion solutions.

The invention will be further explained in more detail by way of examples. These examples are illustrative only and do in no way limit the scope of the invention defined in the claims in view of the contents of the present description.

### Working Examples

### Example 1

### Preparation and Stability of Docetaxel Injection Solution in Ethanol

### Starting Materials:

Ethanol, water content<0.1%
Docetaxel, purity 99.50% (w/w) (determined by high performance liquid chromatography method (HPLC)

### Preparation:

1.0 g docetaxel was dissolved in 25 ml ethanol by stirring at room temperature in absence of light. The resulting solution having the concentration of 40 mg docetaxel per 1 ml of the solution was filtered under sterile conditions through a filter having porosity 0.22 μm. Volumes of 2 ml of the solution were filled into glass (hydrolytic class 1) vials for antibiotics. The vials were closed with teflon coated rubber stoppers and aluminium seals. Each vial contained 80 mg docetaxel.

### Stability Study:

The stability study was performed by subjecting the docetaxel injection solution in ethanol to a temperature of 40° C. at 75% R.H. in absence of light for three months. Docetaxel and related impurities were determined by a slightly adapted HPLC method described in Pharmacopoeial Forum, Vol. 24, No. 6, November-December 1998, p.7167, UV detection 230 nm. The results are summarized in Table 1:

TABLE 1
A three month stability study of docetaxel injection
solution in ethanol, concentration 40 mg docetaxel
per 1 ml ethanol, 40° C. and 75% R.H.

Content (%) after

| Compound | 0 | 1 week | 1 months | 3 months |
|---|---|---|---|---|
| Docetaxel | 99.50 | 99.46 | 99.42 | 99.40 |
| 10-deacetyl | 0.06 | 0.09 | 0.13 | 0.12 |
| baccatin III | | | | |
| Sum of other | 0.44 | 0.45 | 0.45 | 0.48 |
| related impurities | | | | |

The procedure and the results summarized in Table 1 show that docetaxel injection solutions in ethanol are easily prepared and they have very good stability.

### Example 2

### Preparation and Stability of Polysorbate Injection Solution in the Mixture of Ethanol and Water

### Starting Materials:

Polysorbate 80, pharmaceutical grade
Ethanol, water content<0.1%
Water for injection (further abbreviated as WFI)

### Procedure:

40 ml ethanol was dissolved in 80 ml Polysorbate 80 at room temperature and then 120 ml of WFI was added. The resulting polysorbate injection solution comprising ethanol, Polysorbate 80 and WFI at a volume ratio 1:2:3 was filtered under sterile conditions through a filter having porosity 0.22 μm. Volumes of 6 ml of the solution were filled into glass (hydrolytic class 1) vials for antibiotics. The vials were closed with teflon coated rubber stoppers and aluminium seals. Each vial contained 6 ml of the solution comprising 1 ml of ethanol, 2 ml of Polysorbate 80 a and 3 ml of water.

### Stability Study:

The stability study was performed by subjecting the polysorbate injection solution to a temperature of 40° C. at 75% R.H. in absence of light for three months. pH values, acid values, color and clarity were measured. The pH values of 5% (w/w) concentration of Polysorbate 80 after the dilution of the polysorbate injection solution by WFI were measured by a pH-meter. The acid values were measured according to the slightly adapted analytical method described for Polysorbate 80 in US Pharmacopoeia, NF24, p. 3406, wherein 30 ml of the polysorbate injection solution (five vials with polysorbate solution) was diluted by ethanol to a final volume 50 ml before titration. Color and clarity were evaluated visually. The results are summarized in Table 2.

TABLE 2
A three month stability study of polysorbate injection
solution comprising ethanol, Polysorbate 80 and WFI
in a volume ratio 1:2:3, 40° C. and 75% R.H.

Time

| Parameter | 0 | 1 week | 1 month | 3 months |
|---|---|---|---|---|
| Clarity | Clear | Clear | Clear | Clear |
| Color | Near | Near | Near | Near |
| | colorless | colorless | colorless | colorless |
| pH | 6.2 | 6.2 | 6.1 | 6.1 |
| Acid value | 0.83 | 0.83 | 0.88 | 0.87 |

The procedure and the results summarized in the Table 2 show that polysorbate injection solutions comprising ethanol, Polysorbate 80 and WFI are easily prepared and they have very good stability.

### Example 3

### Preparation and Stability of Docetaxel Premix Solution

2 ml docetaxel injection solution in ethanol prepared according to Example 1 and comprising 80 mg of docetaxel was used as solution a).

6 ml polysorbate injection solution prepared according to Example 2 and comprising ethanol, Polysorbate 80 and WFI in a volume ratio 1:2:3 was used as solution b).

### Procedure:

2 ml of solution a) was mixed with 6 ml of solution b) under sterile conditions and homogenised by repeated inversions without shaking. The resulting docetaxel premix solution had a total volume of 8 ml and per 1 ml it contained 10 mg of docetaxel, 0.375 ml of ethanol and 0.250 ml of Polysorbate 80. The docetaxel premix solution was filled into a glass (hydrolytic class 1) vial for antibiotics. The vial was then closed with a teflon coated rubber stopper and an aluminium seal. Five vials with the-docetaxel premix solution were prepared by the same procedure. Each vial contained 80 mg of docetaxel.

### Stability Study:

The stability study was performed by subjecting the docetaxel premix solution to room temperature at 60% R.H. in the absence of light for one month. Docetaxel and related impurities were determined by HPLC method used in Example 1. The results are summarized in Table 3

TABLE 3
A one month stability study of docetaxel premix solution,
concentration 10 mg of docetaxel per 1 ml of the solution,
room temperature and 60% R.H., absence of light.

| | Content (%) after | | |
|---|---|---|---|
| Compound | 0 | 1 week | 1 month |
| Docetaxel | 99.50 | 98.69 | 95.82 |
| 10-deacetyl | 0.06 | 0.69 | 3.10 |
| baccatin III | | | |
| Sum of other | 0.44 | 0.62 | 1.08 |
| related impurities | | | |
| Clarity | Clear | Clear | Clear |

The procedure and the results summarized in Table 3 show that docetaxel premix solutions which are necessary for the preparation of infusion solutions can be easily prepared and they have acceptable short time stability. That is why it is recommended to mix together the taxane derivative solution in ethanol and the polysorbate solution just before the preparation of the infusion solution.

### Example 4

### Preparation and Stability of a Docetaxel Infusion Solution With Polysorbate

### Procedure:

The content of a vial containing 8 ml docetaxel premix solution prepared according to Example 3 and containing 80 mg docetaxel was injected into 250 glass bottle with precharged 72 ml 5% glucose solution for infusion and then mixed manually by a rocking motion of the glass bottle. The resulting solution for infusion had a volume of 80 ml and it contained 1 mg of docetaxel per 1 ml of the infusion solution. The docetaxel solution for infusion was stored in the closed glass bottle at room temperature in absence of light for one week. No precipitation or change in color were observed. The results show that docetaxel solutions for infusion prepared in this way are stable at least for the time period of one week. It is however recommended to use them without delay to reduce the risk of microbial contamination or occasional precipitation of the drug from the diluted solution.

### Example 5

### Preparation of Paclitaxel Injection Solution in Ethanol, Paclitaxel Premix Solution With Polysorbate and Paclitaxel Solution For Infusion With Polysorbate

Paclitaxel having purity 99.73% (w/w) (detemined by HPLC method) was used instead of docetaxel.

### a) Preparation of Paclitaxel Injection Solution in Ethanol

A procedure similar to that of Example 1 was used. 1.0 g paclitaxel was dissolved in 50 ml ethanol to obtain 50 ml paclitaxel injection solution containing 20 mg of paclitaxel per 1 ml of the solution. Volumes 5 ml of the sterile paclitaxel injection solution were filled into glass (hydrolytic class 1) vials for antibiotics. The vials were then closed with teflon coated rubber stoppers and aluminium seals. Each vial contained 100 mg of paclitaxel.

### b) Preparation of Polysorbate Injection Solution.

A procedure similar to that of Example 2 was used. 25 ml ethanol was dissolved in 75 ml Polysorbate 80 at room temperature. Volumes of 10 ml of the sterile polysorbate solution were filled into glass (hydrolytic class 1) vials for antibiotics. The vials were then closed with teflon coated rubber stoppers and aluminium seals.

### c) Preparation of Paclitaxel Premix Solution.

A procedure similar to that of Example 3 was used. 5 ml paclitaxel injection solution in ethanol comprising 100 mg paclitaxel was used as solution a). 10 ml polysorbate injection solution comprising 7.5 ml Polysorbate 80 and 2.5 ml ethanol was used as solution b).

5 ml of paclitaxel solution a) was mixed with 10 ml of the polysorbate solution b) under sterile conditions and the mixture was homogenised by repeated inversions without shaking. The resulting paclitaxel premix solution had a total volume 15 ml and per 1 ml it contained 6.67 mg paclitaxel, 0.50 ml ethanol and 0.50 ml Polysorbate 80. The prepared paclitaxel premix solution was filled into a glass (hydrolytic class 1) vial for antibiotics which was then closed with teflon coated rubber stopper and aluminium seal. The prepared paclitaxel premix solution injection contained 100 mg of paclitaxel. The paclitaxel premix solution injections were stored at room temperature and 60% R.H. in absence of light for one month. No precipitation or change in color was observed.

### d) Preparation of Paclitaxel Solution for Infusion

A procedure similar to that of Example 4 was used. 15 ml of paclitaxel premix solution containing 100 mg of paclitaxel was injected into a 250 ml glass bottle with precharged 85 ml 5% glucose solution for infusion and then it was mixed manually by a rocking motion of the glass bottle. The resulting solution for infusion had a volume of 100 ml and it contained 1 mg of paclitaxel per 1 ml of the solution for infusion. The prepared paclitaxel solution for infusion was stored in the closed glass bottle at room temperature in absence of light for one week. No precipitation or change in color was observed.

### Example 6

### Preparation of Docetaxel Injection Solution in Ethanol, Docetaxel Premix Solution With Polyoxyethyleneglycerol Triricinoleate and Docetaxel Solution For Infusion With Polyoxyethyleneglycerol Triricinoleate

### Starting Materials:

Docetaxel, purity 99.50% (w/w) (determined by HPLC)
Polyoxyethyleneglycerol triricinoleate: Cremophor ELP
Ethanol, water content<0.1%
Water for injection (WFI)

### a) Preparation of Docetaxel Injection Solution in Ethanol

The procedure according to Example 1 was used. Ten vials containing 80 mg of docetaxel, each, were prepared.

### b) Preparation of Polyoxyethyleneglycerol Triricinoleate Injection Solution

The procedure according to example 2 was used with the exception that Cremophor ELP instead of Polysorbate 80 was used as a surfactant. Volumes of 6 ml of the resulting sterile solution were filled into 10 glass (hydrolytic class 1) vials for antibiotics. The vials were then closed with teflon coated rubber stoppers and aluminium seals. Each vial contained ethanol, polyoxyethyleneglycerol triricinoleate and water at a volume ratio 1:2:3. Ten vials were used for the preparation of a premix solutions in the next step.

c) Preparation of Docetaxel Premix Solution With Polyoxyethyleneglycerol Triricinoleate

The procedure according to example 3 was used. Each prepared docetaxel premix solution had a total volume 8 ml and per 1 ml it contained 10 mg docetaxel, 0.375 ml ethanol and 0.250 ml polyoxyethyleneglycerol triricinoleate.

### d) Preparation of Docetaxel Solution For Infusion With Polyoxyethyleneglycerol Triricinoleate

The procedure according to example 4 was used. The resulting solution for infusion had a volume of 80 ml and per 1 ml it contained 1 mg docetaxel and 0.025 ml of polyoxyethyleneglycerol triricinoleate. The infusion solution was stored in the closed glass bottle at room temperature in absence of light for three days. No precipitation or change in color was observed.

1. A two-component taxane containing pharmaceutical composition useful for the preparation of infusion solutions wherein decomposition and/or precipitation of taxane derivatives from the infusion solutions is avoided, said composition comprising a) taxane derivative solution in ethanol and b) a solution of a surfactant selected from polysorbates or polyoxyethylated castor oil (polyoxyethylene-glycerol triricinoleate) in ethanol or a mixture of ethanol and water.

2. The pharmaceutical composition according to claim 1, wherein the taxane derivative solution in ethanol a) is a docetaxel solution in ethanol having a concentration of docetaxel from about 10 to about 100 mg/ml.

3. The pharmaceutical composition according to claim 1, wherein the taxane derivative solution in ethanol a) is a paclitaxel solution in ethanol having a concentration of paclitaxel in the range from about 10 to about 25 mg/ml.

4. The pharmaceutical composition according to claim 1, wherein the surfactant is Polysorbate 80.

5. The pharmaceutical composition according to claim 4, wherein an amount of Polysorbate 80 is the amount necessary to prevent precipitation of a taxane derivative from an infusion solution.

6. (canceled)

7. A method for the preparation, of an infusion solution comprising a taxane derivative, which comprises the steps of
i) mixing a) a taxane derivative solution in ethanol and b) a solution of a surfactant selected from polysorbates or polyoxyethylated castor oil (polyoxyethyleneglycerol triricinoleate) in ethanol or a mixture of ethanol and water and then
ii) diluting the premix from step i) with a common unloaded infusion solution.

8. The method according to claim 7, wherein both steps i) and ii), and in particular step ii), are carried out shortly before using the infusion solution prepared in claim 7.

9. The composition according to claim 2, wherein the concentration of docetaxel is about 40 to about 80 mg/ml.

10. The composition according to claim 3, wherein the concentration of paclitaxel is about 15 to about 20 mg/ml.

11. A method for treating cancer comprising administering a composition according to claim 1 to a subject in need thereof.
